(19) United States
(12) Patent Application Publication
(10) Pub. No.: US 2014/0171716 A1
(43) Pub. Date: Jun. 19, 2014

(54) SEPARATION OF IMPURITIES FROM A HYDROCARBON-CONTAINING GAS STREAM

(71) Applicant: UOP LLC, Des Plaines, IL (US)

(72) Inventors: Stanislav Milidovich, Gurnee, IL (US); Ernest James Boehm, Hanover Park, IL (US); William J. Lechnick, Glen Ellyn, IL (US)

(73) Assignee: UOP LLC, Des Plaines, IL (US)

(21) Appl. No.: 13/715,785

(22) Filed: Dec. 14, 2012

(51) Int. Cl. C07C 7/00

(52) U.S. Cl. CPC C07C 7/005
USPC 585/802; 96/234

(57) ABSTRACT

A process includes contacting a feed stream of light hydrocarbons and an acid gas with a first solvent stream to produce a first methane-enriched overhead gas stream and a first stage solvent effluent bottoms stream including absorbed methane and absorbed acid gas. The process further includes flash separating the first stage solvent effluent bottoms stream to produce a vapor fraction including the acid gas and methane a gas-depleted liquid fraction of the first stage solvent effluent bottoms stream. The process further includes contacting the first methane-enriched overhead gas stream with a second solvent stream to produce a second methane-enriched overhead gas stream and a second stage solvent effluent bottoms stream including absorbed methane CO₂. Still further, the process includes regenerating the second stage solvent effluent bottoms stream to produce a vapor fraction comprising the acid gas and a regenerated liquid fraction of the second stage solvent effluent bottoms stream.

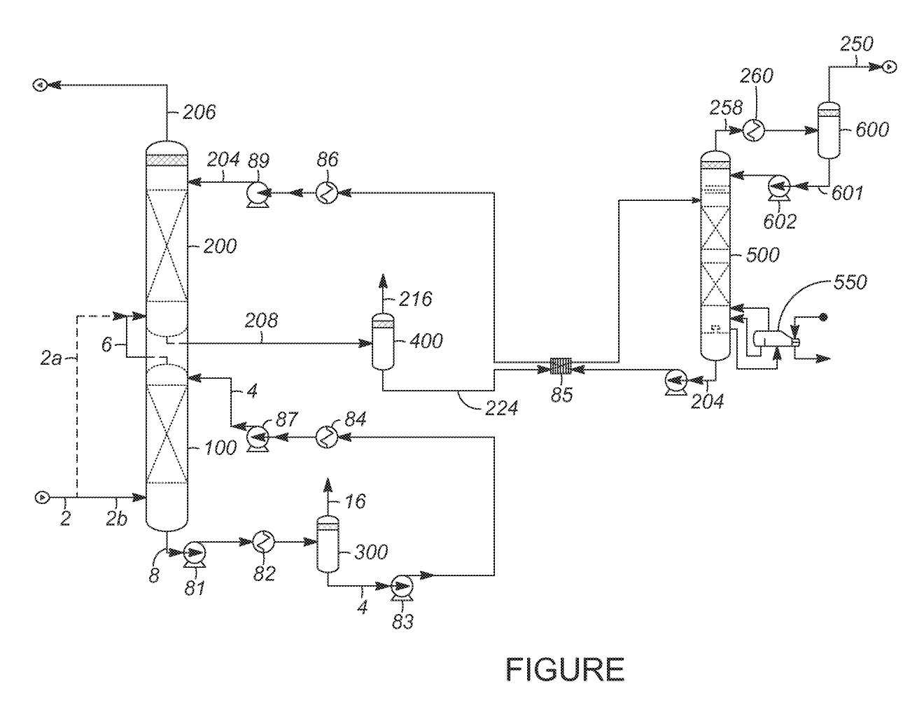

### TECHNICAL FIELD

The present disclosure generally relates to systems and methods for gas component separation. More particularly, the present disclosure relates to systems and methods for the separation of contaminants, such as carbon dioxide (CO₂) and hydrogen sulfide (H₂S), from light hydrocarbon gas streams (e.g. conventional or unconventional natural gas or methane) using a multi-solvent absorption process.

### BACKGROUND

The removal of carbon dioxide (CO₂), hydrogen sulfide (H₂S), and other sulfur bearing species such as mercaptains and carbonyl sulfides, also known as acid gas components, from predominantly light hydrocarbon streams is required in order to meet purified gas product specifications prior to pipeline transmission or liquefaction. Differences in a number of chemical and physical properties between acid gas components and light hydrocarbons can serve as potential bases for gas separations. These differences include solubility, acidity in aqueous solution, and molecular size and structure. Possible separations can therefore rely on physical or chemical absorption into liquid solvents, pressure swing adsorption with solid adsorbents, and membrane systems.

Liquid solvent absorption (i.e., “wet”) systems, for example, are commonly used for natural gas purification to remove minor amounts of acid gas components. These contaminants can be preferentially absorbed in physical solvents such as dimethylethers of polyethylene glycol or chemical solvents such as alkanolamines or alkali metal salts. The resulting acid gas-rich (i.e., “loaded”) solvent is subsequently regenerated by heating to recover the acid gas and a regenerated solvent that can be recycled for further use in absorption. Solvent regeneration can also be conducted by reducing pressure relative to the upstream absorption pressure, to promote vaporization of absorbed acid gas components from the solvent. The solvent absorption and solvent regeneration by heating are usually carried out in different columns containing packing, trays, or other vapor-liquid contacting devices to improve the efficiency of mass transfer between phases. The captured acid gas components may be recovered in more than one stream, including vapor fractions of flash separators and regenerator column vapor effluents.

Chemical solvents, and particularly amines and other basic compounds, react with acid gas contaminants to form a contaminant-solvent chemical bond. Considerable energy release is associated with this bond formation during the thermodynamically-favored, acid-base reaction. Due to the substantial heat input required to break the contaminant-solvent bonds formed during chemical reaction with the solvent and acid gas contaminants, chemical solvents that are thermally regenerated are less economical when comparing to solvents that are regenerated by reduction in pressure. Physical solvents, on the other hand, do not react chemically with gas contaminants, but instead promote physical absorption based on a higher contaminant equilibrium solubility at its partial pressure in the impure gas (i.e., a higher Henry's law constant).

Physical solvents that remain chemically non-reactive with the impure gas stream are therefore desirable in absorption systems due to the ease of solvent regeneration. However, the removal of acid gas contaminants using a physical solvent, especially in rich natural gas feed stocks, is not desired due to co-absorption of other hydrocarbon components. As a result, the recovery of the acid gas contaminants in a purified form remains problematic since the produced acid gas is now contaminated with other hydrocarbons, which causes issues in the downstream operation (e.g., an associated sulfur recovery unit).

Furthermore, impure light hydrocarbon-containing gas streams can vary greatly in the amount of acid gas impurities, for example CO₂, contained in the gas stream. Using the example of natural gas, when a natural gas source is first discovered, the impurity content thereof may be relatively low, for example less than 1 mol % of impurities such as CO₂. As the gas source is developed over time, however, the purity of the gas source may decrease, and the impurity content thereof can increase, for example to greater than 3 mol %.

As such, it will be appreciated that in many instances, a natural gas producer may require natural gas treating for CO₂ removal from a feedstock that may contain 1 mol % CO₂ or less, in order to meet the relevant CO₂ specification required by the liquefaction process used for the production of liquid natural gas (LNG) (for example, less than or equal to about 50 ppm). Over time, however, the same natural gas producer may require natural gas treating for CO₂ removal from a feedstock that may contain 3 mol % CO₂ or greater. In these cases, initially (i.e., while the impurity content is at 1 mol % CO₂ or less) the treating unit, if designed for the eventual use at 3 mol % CO₂, will be grossly over-designed especially if the measured CO₂ concentration will be less than 1 mol % for a significant period of time. Many natural gas producers would not want to pre-invest into a very large treating unit when the need therefore will not be until sometime in the distant future.

Accordingly, it would be an advance in the state of the art to provide systems and methods for acid gas removal from a natural gas flow stream that are able to treat streams that have varying impurity contents. It would further be desirable to provide such systems and methods that are expandable to treating increasing impurity concentrations in the natural gas flow stream over time. Still further, other desirable features and characteristics of the inventive subject matter will become apparent from the subsequent detailed description of the inventive subject matter and the appended claims, taken in conjunction with the accompanying drawings and this background of the inventive subject matter.

### BRIEF SUMMARY

Systems and processes for gas separation are provided herein. In an exemplary embodiment, a two-stage, multi-solvent gas purification process includes contacting an impure feed gas stream of light hydrocarbons, including natural gas or methane, and an acid gas, including CO₂ or H₂S, with a first chemical solvent stream to produce a first semi-purified, methane-enriched overhead gas stream and a first stage solvent effluent bottoms stream including absorbed methane and absorbed acid gas. The process further includes flash separating the first stage solvent effluent bottoms stream to produce (i) a vapor fraction comprising the acid gas and methane and (ii) a gas-depleted liquid fraction of the first stage solvent effluent bottoms stream. The process further includes contacting the first semi-purified, methane-enriched overhead gas stream with a second chemical solvent stream to produce a second purified, methane-enriched overhead gas stream and a second stage solvent effluent bottoms stream including absorbed methane and absorbed CO₂. Still further, the process includes regenerating the second stage solvent effluent bottoms stream to produce (i) a vapor fraction comprising the acid gas and (ii) a gas-depleted, regenerated liquid fraction of the second stage solvent effluent bottoms stream.

In another exemplary embodiment, a two-stage, multi-solvent gas purification system includes a first counter-current absorber configured to contact an impure feed gas stream comprising light hydrocarbons, including natural gas or methane, and an acid gas, including CO₂ or H₂S, with a first chemical solvent stream in a first counter-current absorber to produce a first semi-purified, methane-enriched overhead gas stream and a first stage solvent effluent bottoms stream including absorbed methane and absorbed acid gas. The system further includes a flash separator configured to flash separate the first stage solvent effluent bottoms stream to produce (i) a vapor fraction including the acid gas and methane and (ii) a gas-depleted liquid fraction of the first stage solvent effluent bottoms stream. The system further includes a second counter-current absorber configured to contact the first semi-purified, methane-enriched overhead gas stream with a second chemical solvent stream to produce a second purified, methane-enriched overhead gas stream and a second stage solvent effluent bottoms stream comprising absorbed methane and absorbed CO₂. Still further, the system includes a solvent regenerator configured to regenerate the second stage solvent effluent bottoms stream to produce (i) a vapor fraction including the acid gas and (ii) a gas-depleted, regenerated liquid fraction of the second stage solvent effluent bottoms stream.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### BRIEF DESCRIPTION OF THE DRAWING

The systems and methods of the present disclosure will hereinafter be described in conjunction with the FIGURE, which is a diagram of one embodiment of a system employing a process for carbon dioxide removal from a natural gas flow stream.

### DETAILED DESCRIPTION

The following detailed description is merely exemplary in nature and is not intended to limit the disclosure or the application and uses of the illustrated embodiments. All of the embodiments and implementations described herein are exemplary embodiments provided to enable persons skilled in the art to make or use the invention and not to limit the scope of the invention, which is defined by the claims. Furthermore, there is no intention to be bound by any expressed or implied theory presented in the preceding technical field, background, brief summary, or the following detailed description.

Embodiments of the present disclosure are directed to gas purification systems and methods in which a contaminant, such as CO₂, present as a minor component of an impure feed gas, is selectively absorbed into a solvent. Representative impure gas streams include those having light hydrocarbons (e.g., C₁-C₃ hydrocarbons), such as natural gas, and non-hydrocarbon gas contaminants, such as carbon dioxide (CO₂). Examples of such gas streams include natural gas and coal-bed methane, which include predominantly methane and also include CO₂ in an amount from about 1 mol % to about 10 mol % by volume. For simplicity, the illustrative embodiments are described hereinafter with respect to such methane and CO₂ systems with the latter component being present at contaminant levels, although it will be appreciated that the invention is broadly applicable to the purification of impure gas feeds in which a contaminant, present in an minor amount, is preferentially absorbed into a liquid solvent, and particularly a physical solvent.

Representative embodiments are directed to gas purification systems and methods involving several separation stages. In one stage, referred to herein as an absorption contacting stage, an impure feed gas is contacted with a solvent. There may be one, two, or more such absorption contacting stages, each such stage using the same or a different solvent. In another contacting stage, referred to herein as a “regeneration” stage, the “loaded” solvent from the absorption contacting stage or stages, including absorbed components of the impure feed gas, is subjected to regeneration, meaning that a vapor is introduced to drive off at least some of these absorbed components, into a gas effluent from the regeneration stage. The absorption and regeneration stages are normally each carried out in separate absorber and regeneration vessels, with liquid solvent streams flowing downwardly toward a solvent effluent outlet, counter-current to gas streams flowing upwardly toward a gas effluent outlet. It is, however, in alternate embodiments, possible for absorption and regeneration to be performed in a single vessel, such as in a stacked arrangement in upper and lower sections of the vessel, respectively.

A gas purification method according to an exemplary embodiment therefore includes contacting an impure feed gas including methane and CO₂ with a solvent, particularly a chemical solvent that selectively (or preferentially) absorbs CO₂ over methane. Representative chemical solvents include, for example, the various UCARSOL™ AP-800 series solvents manufactured by The Dow Chemical Company of Midland, Mich., USA. UCARSOL™ AP-800 solvents are advanced-performance gas-treating solvents designed to remove carbon dioxide (CO₂), hydrogen sulfide (H₂S), and/or carbonyl sulfide (COS) from natural and synthesis gas. These solvents are methyl-diethanolamine (MDEA) based solvents with low concentrations of an activator compound that accelerates the slow reaction kinetics of CO₂ with the MDEA. The feed gas-solvent contacting in the absorption contacting stage(s) therefore provides (i) a gas with a higher methane content than the feed gas (i.e., a methane-enriched gas) and (ii) a solvent effluent comprising the absorbed CO₂ as well as some absorbed methane, with (i) and (ii) exiting the first contacting stage as vapor and liquid phases, respectively.

Embodiments of the present disclosure may further, optionally, employ one or more flash separation stages subsequent to the one or more absorption contacting stages. Flash separation is obtained by the vaporization of some portion of the gases absorbed into the liquid solvent effluent exiting the absorption contacting stage(s) as a liquid phase. This vaporization may be achieved by depressurization and/or heating of the absorption separation stage(s) solvent effluent. In the case of a plurality of flash separations (e.g., the use of two flash separations in series), successively lower pressure flash separations (e.g., at high pressure followed by lower pressure) may be utilized to provide a plurality of vapor fractions, all or a portion of which may be combined to provide the combined vapor fraction.

The solvent effluent is then passed through a regeneration stage of separation. The regeneration stage solvent effluent is obtained as a liquid phase exiting this stage. The regeneration stage also generally provides an acid gas (i.e., having a higher CO₂ content than the vapor fraction), that exits the regeneration stage as a vapor phase.

More particularly, because CO₂ is preferentially absorbed in the absorption solvent over methane, the absorbed gases remaining in the gas-depleted liquid fraction or solvent phase, after the removal of the vapor fraction via optional flash separation, are enriched in CO₂. Therefore, the purified CO₂ remaining in the gas-depleted liquid fraction is advantageously recovered as a product gas, in addition to the purified methane product gas as discussed above. Often, the purified CO₂ product gas is recovered after heating at least a portion of the gas-depleted liquid fraction of the solvent effluent, as necessary to regenerate the solvent. The purified CO₂ product gas recovered from all or a portion of the gas-depleted liquid fraction, according to representative embodiments of the disclosure, includes CO₂ in an amount of generally at least about 90% (e.g., in the range from about 90% to about 99%) by volume, typically at least about 92% (e.g., in the range from about 92% to about 99%) by volume, and often at least about 94% (e.g., in the range from about 94% to about 99%) by volume, on a water-free basis. In a specific embodiment, for example, the portion of the gas-depleted liquid fraction is obtained after flash separation where a CO₂-rich solvent stream, which is a liquid fraction, is flash separation (e.g., at low pressure) to provide a vapor fraction, which is mostly CO₂, and a lean liquid fraction that is recycled to the first contacting stage.

Beneficially, the various stages of the separation system of the present disclosure are configured so as to accommodate gas streams of various impurity levels, and are also configured so as to accommodate gas streams that have impurity levels that change over time. In a particular embodiment, the separation system includes an absorption separation stage, followed by a flash separation stage, followed by a solvent regeneration stage. This particular embodiment further includes a second absorption separation stage followed by a second flash separation stage. The solvent in the first absorption separation stage is configured to reduce CO₂ impurities in the feed gas from about 1 mol % to about 50 ppm. In this manner, the first absorption separation stage is configured to process relatively pure feed gas streams, such as may be encountered in newly recovered natural gas streams. The solvent in the second absorption separation stage is configured to reduce CO2 impurities in the feed gas from about 3 mol % to about 1 mol %. In this manner, the second absorption separation stage is configured to process relatively impure feed gas streams, such as may be encountered in natural gas streams in later years of recovery. The second absorption separation stage may be bypassed, if initially provided along with the first absorption separation stage, for feed gas streams with 1 mol % of CO₂ impurities or less. If at a later date the feed gas increases impurities, the feed gas may be first passed through the second absorption separation stage to reduce the relatively impure feed gas to 1 mol % or less of CO₂, and then passed to the first absorption separation stage. Alternatively, the separation system may be initially provided with only the first absorption separation system while the feed gas stream is relatively pure, and then at a later date the second absorption separation system may be added as the feed gas becomes more impure over time. In this manner, a large initial capital outlay and/or oversized separation system may be avoided by light hydrocarbon gas processors, thereby reducing the costs associated with processing the gas.

An exemplary, two-stage, gas purification process according to an embodiment as described herein is illustrated in THE FIGURE. As shown, an impure feed gas stream 2 including methane and impurity, for example CO₂, is provided. Where only a single absorption separation is desired, as in a feed gas with less than or equal to about 1 mol % CO₂, the impure feed gas stream 2 continues to bypass stream 2a. Where two absorption separations are desired, as in a feed gas with greater than 1 mol % CO₂, the feed stream continues to primary stream 2b.

In a two-stage process, primary stream 2b is contacted with solvent stream 4 in counter-current absorber 100 to provide, as exiting vapor and liquid phases, methane-enriched overhead gas stream 6 and first stage solvent effluent bottoms stream 8 including absorbed portions of methane and CO₂. The overhead gas stream 6 has been reduced in CO₂ content from as great as about 3 mol % to about 1 mol %. A suitable absorption solvent to achieve such reduction in CO₂ content is, as noted above, one of the UCARSOL™ AP-800 series solvents. In a particular embodiment, the solvent is the AP-802 solvent.

The bottoms stream 8 optionally flows to a solvent power recovery turbine, wherein energy is recovered from the flowing CO₂-rich solvent stream. Thereafter, stream 8 is heated in a heater, prior to entry into flash separator 300. Flash separation of solvent effluent bottoms stream 8 in high pressure flash separator 300 provides a high-pressure vapor fraction 16, which includes CO₂ and some of the small fraction of methane that was absorbed into the solvent. The high-pressure flash separator 300 operates generally at a pressure of less than about 2.9×10⁶ Pa (27 barg; 400 psig) (e.g., in the range from about 2.4×10⁵ Pa (2 barg; 30 psig) to about 2.9×10⁶ Pa (27 barg; 400 psig)). For example, high pressure flash separator 300 may be maintained at a pressure in the range from about 2.2×10⁶ Pa (21 barg; 300 psig) to about 2.9×10⁶ Pa (27 barg; 400 psig).

As a result of the flash separation to provide vapor fraction 16, corresponding liquid solvent fraction 4 is also provided. Corresponding liquid fraction 4 is referred to as a semi-lean solvent, because some, but not all, of the CO₂ and small portion of methane absorbed therein in the absorption separator 100 has been removed by means of the flash separator 100. The semi-lean solvent stream 4 is then pumped via pump 83 to a solution cooler 84, wherein the semi-lean solvent is cooled to an appropriate temperature for re-entry into the overhead portion of the absorption separator 100 via pump 87. In general, semi-lean solvent stream 4 is combined with an optional make-up solvent stream to provide solvent that is introduced to counter-current absorber 100 as described above. Optional make-up solvent stream replaces the total solvent losses throughout the gas purification process.

As noted above, overhead gas stream 6 has been reduced in CO₂ content to less than or equal to about 1 mol %. Overhead gas stream 6 is thereafter contacted with solvent stream 204 in counter-current absorber 200 to provide, as exiting vapor and liquid phases, methane-enriched overhead gas stream 206 and second stage solvent effluent bottoms stream 208 including absorbed portions of methane and CO₂. Of course, where the feed gas stream 2 was bypassed into bypass stream 2a, for example where the feed gas stream 2 included less than or equal to about 1 mol % of CO₂, bypass stream 2a would be contacted with solvent stream 204 in the absorber 200. The overhead gas stream 206 has been reduced in CO₂ content from as great as about 1 mol % to about 50 ppm. A suitable absorption solvent to achieve such reduction in CO₂ content is, as noted above, one of the UCARSOL™ AP-800 series solvents. In a particular embodiment, the solvent is the AP-814 solvent.

As shown in THE FIGURE, the absorption separators 100, 200 can be configured in a “stacked” design, one on top of the other. This particular design is anticipated to allow for easy expansion of the unit when needed and reduced energy costs when both sections 100, 200 are operated (where the feed stream is greater than 1 mol % CO₂).

The bottoms stream 208 thereafter enters into flash separator 400. Flash separation of solvent effluent bottoms stream 208 in high pressure flash separator 400 provides a high-pressure vapor fraction 216, which includes CO₂ and some of the small fraction of methane that was absorbed into the solvent. The high-pressure flash separator 400 operates generally at a pressure of less than about 2.9×10⁶ Pa (27 barg; 400 psig) (e.g., in the range from about 2.4×10⁵ Pa (2 barg; 30 psig) to about 2.9×10⁶ Pa (27 barg; 400 psig)). For example, high pressure flash separator 300 may be maintained at a pressure in the range from about 2.2×10⁶ Pa (21 barg; 300 psig) to about 2.9×10⁶ Pa (27 barg; 400 psig).

As a result of the flash separation to provide vapor fraction 216, corresponding liquid solvent fraction 224 is also provided. Corresponding liquid fraction 224 is referred to as a semi-lean solvent, because some, but not all, of the CO₂ and small portion of methane absorbed therein in the absorption separator 400 has been removed by means of the flash separator 200.

An acid gas stream 250 including primarily CO₂ gas is recovered from stream 224, for example, by heating at least a portion of this stream to regenerate the solvent. In the specific embodiment illustrated in THE FIGURE, for example, solvent stream 224 is passed to rich/lean heat exchanger 85 of solvent regenerator 500. Rich/lean heat exchanger 85 is used in conjunction with solvent reboiler 550 and optionally one or more heaters of solvent regenerator 500, to heat the solvent stream 224 of the CO₂-rich solvent. This CO₂-rich solvent portion 224, as an inlet to solvent regenerator 500, is typically heated to a temperature in the range from about 38° C. (100° F.) to about 204° C. (400° F.), and often in the range from about 66° C. (150° F.) to about 149° C. (300° F.), depending on the particular solvent and component(s) of impure gas stream 2 to be recovered. In general, the solvent stream 224 is heated to a temperature of at least about 150° C. (302° F.) to regenerate the solvent and recover CO₂ acid gas stream 250.

In the exemplary embodiment depicted in THE FIGURE, CO₂ acid gas stream 250 is recovered from overhead vapor stream 258 of solvent regenerator 500, generally after this stream is passed through an overhead condenser 260 and overhead reflux drum 600 to which a make-up water stream. The reflux drum 600 bottoms stream 601 is passed to reflux pump 602, for re-entry back into the overhead of the solvent regenerator 500.

Regenerated solvent stream 204 is recovered from a bottom section of solvent regenerator 500, substantially depleted of all absorbed gases, and may be cooled by heat exchange against CO₂-rich solvent portion 224 using heat exchanger 85 and further cooled using a solution cooler 89 prior to introduction into counter-current absorber 200 via pump 86. In general, regenerated solvent stream 204 is combined with an optional make-up solvent stream to provide solvent that is introduced to counter-current absorber 200 as described above. The optional make-up solvent stream replaces the total solvent losses throughout the gas purification process.

Accordingly, an improved system and method for carbon dioxide removal from a natural gas stream has been described. It will be appreciated that the described systems and methods are ideal for natural gas processors who are looking at treating pipeline natural gas to LNG specifications. As will be appreciated, most of the time the CO₂ concentration in natural gas pipeline is relatively low (less than 1 mol %); however, the pipeline design specification may call for CO₂ concentrations of as high as 3 mol %. With this design, the natural gas processor, who may only see about 1 mol % CO₂ in their pipeline gas, may chose to pre-invest into the first absorption separation section while leaving out all other equipment required for the second absorption separation section. When or if the CO₂ concentration becomes higher (greater than 1 mol %), the processor can add the additional equipment necessary to handle the increased CO₂ concentration. These changes would be relatively easy without major modifications to the existing unit and at relatively low expansion cost. Further, the single unit design of the absorption separators optimizes the energy demand for each section and allows lower turndown ratios with respect to CO₂ concentration in the feed.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the processes without departing from the scope defined by the claims, which includes known equivalents and foreseeable equivalents at the time of this disclosure.

1. A two-stage, multi-solvent gas purification process comprising:
contacting an impure feed gas stream comprising light hydrocarbons, including natural gas or methane, and an acid gas, including CO₂ or H₂S, with a first chemical solvent stream to produce a first semi-purified, methane-enriched overhead gas stream and a first stage solvent effluent bottoms stream comprising absorbed methane and absorbed acid gas;
flash separating the first stage solvent effluent bottoms stream to produce (i) a vapor fraction comprising the acid gas and methane and (ii) a gas-depleted liquid fraction of the first stage solvent effluent bottoms stream;
contacting the first semi-purified, methane-enriched overhead gas stream with a second chemical solvent stream to produce a second purified, methane-enriched overhead gas stream and a second stage solvent effluent bottoms stream comprising absorbed methane and absorbed CO₂; and
regenerating the second stage solvent effluent bottoms stream to produce (i) a vapor fraction comprising the acid gas and (ii) a gas-depleted, regenerated liquid fraction of the second stage solvent effluent bottoms stream.

2. The process of claim 1, wherein the first chemical solvent stream and the second chemical solvent stream comprise methyl-diethanolamine based solvents.

3. The process of claim 2, wherein the first chemical solvent stream and the second chemical solvent stream comprise different solvents.

4. The process of claim 1, wherein the impure feed gas stream comprises CO₂.

5. The process of claim 1, wherein the impure feed gas stream comprises H₂S.

6. The process of claim 1, further comprising flash-separating the second stage solvent effluent bottoms stream.

7. The process of claim 6, further comprising flash-separating the second stage solvent effluent bottoms stream to produce (i) a vapor fraction comprising the acid gas and methane and (ii) a gas-depleted liquid fraction of the second stage solvent effluent bottoms stream.

8. The process of claim 7, wherein regenerating the second stage solvent effluent bottoms stream comprises regenerating the gas-depleted liquid fraction of the second stage solvent effluent bottoms stream.

9. The process of claim 1, further comprising feeding a make-up to combine with the gas-depleted, regenerated liquid fraction of the second stage solvent effluent bottoms stream.

10. A two-stage, multi-solvent gas purification system comprising:
a first counter-current absorber configured to contact an impure feed gas stream comprising light hydrocarbons, including natural gas or methane, and an acid gas, including CO₂ or H₂S, with a first chemical solvent stream in a first counter-current absorber to produce a first semi-purified, methane-enriched overhead gas stream and a first stage solvent effluent bottoms stream comprising absorbed methane and absorbed acid gas;
a flash separator configured to flash separate the first stage solvent effluent bottoms stream to produce (i) a vapor fraction comprising the acid gas and methane and (ii) a gas-depleted liquid fraction of the first stage solvent effluent bottoms stream;
a second counter-current absorber configured to contact the first semi-purified, methane-enriched overhead gas stream with a second chemical solvent stream to produce a second purified, methane-enriched overhead gas stream and a second stage solvent effluent bottoms stream comprising absorbed methane and absorbed CO₂; and
a solvent regenerator configured to regenerate the second stage solvent effluent bottoms stream to produce (i) a vapor fraction comprising the acid gas and (ii) a gas-depleted, regenerated liquid fraction of the second stage solvent effluent bottoms stream.

11. The system of claim 10, wherein the first chemical solvent stream and the second chemical solvent stream comprise methyl-diethanolamine based solvents.

12. The system of claim 11, wherein the first chemical solvent stream and the second chemical solvent stream comprise different solvents.

13. The system of claim 10, wherein the impure feed gas stream comprises CO₂.

14. The system of claim 10, wherein the impure feed gas stream comprises H₂S.

15. The system of claim 10, further comprising a second flash separator configured to flash-separate the second stage solvent effluent bottoms stream to produce (i) a vapor fraction comprising the acid gas and methane and (ii) a gas-depleted liquid fraction of the second stage solvent effluent bottoms stream.

16. The system of claim 10, wherein the first counter-current absorber and the second counter-current absorber are provided in a stacked configuration.

17. A two-stage, multi-solvent gas purification process comprising:
contacting an impure feed gas stream comprising light hydrocarbons, including natural gas or methane, and an acid gas, including CO₂ or H₂S, with a first chemical solvent stream in a first counter-current absorber to produce a first semi-purified, methane-enriched overhead gas stream and a first stage solvent effluent bottoms stream comprising absorbed methane and absorbed acid gas;
flash separating the first stage solvent effluent bottoms stream in a flash-separator to produce (i) a vapor fraction comprising the acid gas and methane and (ii) a gas-depleted liquid fraction of the first stage solvent effluent bottoms stream;
contacting the first semi-purified, methane-enriched overhead gas stream with a second chemical solvent stream in a second counter-current absorber to produce a second purified, methane-enriched overhead gas stream and a second stage solvent effluent bottoms stream comprising absorbed methane and absorbed CO₂; and
regenerating the second stage solvent effluent bottoms stream in a solvent regenerator to produce (i) a vapor fraction comprising the acid gas and (ii) a gas-depleted, regenerated liquid fraction of the second stage solvent effluent bottoms stream.

18. The process of claim 17, further comprising flash-separating the second stage solvent effluent bottoms stream in a second flash separator to produce (i) a vapor fraction comprising the acid gas and methane and (ii) a gas-depleted liquid fraction of the second stage solvent effluent bottoms stream.

19. The process of claim 18, wherein regenerating the second stage solvent effluent bottoms stream comprises regenerating the gas-depleted liquid fraction of the second stage solvent effluent bottoms stream.

20. The process of claim 17, wherein the first counter-current absorber and the second counter-current absorber are provided in a stacked configuration.
